(19) United States
(12) Patent Application Publication
(10) Pub. No.: US 2024/0108356 A1
(43) Pub. Date: Apr. 4, 2024

(54) LOCKING FEATURE FOR HEMOSTASIS CLIP

(71) Applicant: BOSTON SCIENTIFIC SCIMED, INC., Maple Grove, MN (US)

(72) Inventors: Jimena CESPEDES BERROCAL, Alajuela (CR); Edgardo Andres VELÁZQUEZ-RAMÍREZ, Brookline, MA (US); Joseph W. KING, Franklin, MA (US); Alex ROBERTS, Marlborough, MA (US); Juan Pablo ORTIZ GARCIA, Heredia (CR)

(21) Appl. No.: 18/466,284

(22) Filed: Sep. 13, 2023

Related U.S. Application Data
(60) Provisional application No. 63/377,260, filed on Sep. 27, 2022.

(51) Int. Cl. A61B 17/128; A61B 17/122

(52) U.S. Cl. CPC A61B 17/1285; A61B 17/122; A61B 2017/12004

(57) ABSTRACT

An applicator of a clip system includes an elongated member and a bushing coupled to a distal end of the elongated member and a coupling movably attached to the bushing. A capsule is coupled to the coupling via a coupling member hooked over a hook of the coupling. Proximal ends of clip arms are received within the capsule and a core member received between and connected to the proximal ends of the arms includes a projection and a failure point distal of the projection. The core member is coupled to a control member so that movement of the control member relative to the elongated member opens and closes the arms. The projection is configured so that, when the clip is clipped over tissue, the coupling is drawn into the body and the projection drives the coupling member radially outward off the hook to free the capsule.

### PRIORITY CLAIM

The present disclosure claims priority to U.S. Provisional Patent Application Ser. No. 63/377,260 filed Sep. 27, 2022; the disclosure of which is incorporated herewith by reference.

### FIELD

The present disclosure relates to endoscopic devices and, in particular, relates to endoscopic clipping devices for treating tissue along the gastrointestinal tract.

### BACKGROUND

It is often necessary to close tissue openings in gastrointestinal (GI) procedures or for procedures in other body lumens. Currently, such openings are often closed using hemostatic clips. However, certain of these clips generate shed parts upon deployment tissue that may become lodged within the tissue opening being treated or which may be otherwise harmful to the patient.

### SUMMARY

The present disclosure relates to a clipping system for treating tissue. The system includes an applicator, a clip and a core member. The applicator includes a flexible elongate member extending from a proximal end which, during use, remains outside a living body to a distal end which, during use, is inserted into the living body to a location adjacent to target tissue to be clipped. The applicator includes a control member extending through the applicator; and a bushing coupled to a distal end of the elongate flexible member, the bushing including a proximal body coupled to a distal end of the flexible elongate member and a distal coupling movably attached to the proximal body.

The clip includes a capsule releasably coupled to the distal coupling of the bushing via a first coupling member of the capsule hooked over a corresponding first hook of the distal coupling of the bushing. The capsule includes a channel extending therethrough. The clip also includes a pair of clip arms, proximal ends of which are slidably received within the channel for movement between an open configuration, in which distal ends of the clip arms are separated from one another to receive tissue therebetween, and a closed configuration, in which the distal ends of the clip arms are drawn toward one another to grip tissue.

The core member is received between and connected to the proximal ends of the clip arms and including a first lateral projection and a first failure point distal of the first lateral projection. The first failure point is separating a distal portion of the core member from a proximal portion thereof. The core member is coupled to a distal end of the control member so that longitudinal movement of the control member relative to the elongate flexible member moves the clip arms between the open configuration and the closed configuration, a lateral extent of the first lateral projection being selected so that, when the clip is clipped over target tissue, the first lateral projection is drawn proximally moving the distal coupling into the proximal body of the bushing, the first lateral projection driving the first coupling member radially outward beyond an outer end of the first hook to free the capsule from the bushing.

In an embodiment, the core member includes a first pin received within a hole in a proximal part of a first one of the clip arms and a second pin received within a hole in a proximal part of a second one of the clip arms.

In an embodiment, the distal coupling is slidably coupled to the proximal body of the bushing for movement proximally and distally into and out of a distal end of the proximal body.

In an embodiment, the proximal body of the bushing includes a proximal section having an outer diameter substantially equal to an outer diameter of the flexible elongate member and a distal section of the proximal body of the bushing has an outer diameter substantially equal to an outer diameter of the capsule.

In an embodiment, an inner diameter of the channel is substantially equal to an inner diameter of a distal section of the proximal body of the bushing.

In an embodiment, the lateral extent of the first lateral projection is selected to be substantially equal to the inner diameters of the channel and the distal section of the proximal body of the bushing and wherein a proximal portion of the distal coupling has an inner diameter slightly smaller than that of the channel and the distal section of the proximal body of the bushing so that the first lateral projection becomes lodged against the distal coupling within the bushing.

In an embodiment, the core member includes a second failure point proximal of the first failure point, the core member defining the distal portion distal of the first failure point, a medial portion between the first and second failure points and the proximal section proximal of the second failure point.

In an embodiment, the clip arms are constructed to define a proximal-most position of the clip arms within the capsule so that, when a proximally tension applied to the control member after the clip arms have reached the proximal-most position exceeds a first threshold level, the core member separates at the first failure point permitting the medial and proximal portions of the core member to move proximally drawing the first lateral projection proximally out of the capsule driving the first coupling member of the capsule radially outward beyond an outer end of the first hook of the distal coupling of the bushing to separate the capsule from the bushing.

The present disclosure also relates to a device for clipping tissue. The device includes a bushing including a proximal body configured to be coupled to a distal end of a flexible applicator and a distal coupling movably attached to the proximal body, the distal coupling including a first hook. The device also includes a clip including a capsule releasably coupled to the distal coupling via a first coupling member of the capsule hooked over the first hook, the capsule including a channel extending therethrough, and a pair of clip arms, proximal ends of which are slidably received within the channel for movement between an open configuration, in which distal ends of the clip arms are separated from one another to receive tissue therebetween, and a closed configuration, in which the distal ends of the clip arms are drawn toward one another to grip tissue. In addition, the device includes a core member received between and connected to the proximal ends of the clip arms and including a first lateral projection and a first failure point distal of the first lateral projection, the first failure point separating a distal portion of the core member from a proximal portion thereof, the core member configured to be coupled to a distal end of a control member so that longitudinal movement of the control member relative to the flexible applicator moves the clip arms between the open configuration and the closed configuration, a lateral extent of the first lateral projection being selected so that, when the clip is clipped over target tissue, the first lateral projection is drawn proximally moving the distal coupling into the proximal body of the bushing, the first lateral projection driving the first coupling member radially outward beyond an outer end of the first hook to free the capsule from the bushing.

In an embodiment, the core member includes a first pin received within a hole in a proximal part of a first one of the clip arms and a second pin received within a hole in a proximal part of a second one of the clip arms.

In an embodiment, the distal coupling is slidably coupled to the proximal body of the bushing for movement proximally and distally into and out of a distal end of the proximal body.

In an embodiment, the proximal body of the bushing includes a proximal section having an outer diameter substantially equal to an outer diameter of the flexible applicator and a distal section of the proximal body of the bushing has an outer diameter substantially equal to an outer diameter of the capsule.

In an embodiment, an inner diameter of the channel is substantially equal to an inner diameter of a distal section of the proximal body of the bushing.

In an embodiment, the lateral extent of the first lateral projection is selected to be substantially equal to the inner diameters of the channel and the distal section of the proximal body of the bushing and wherein a proximal portion of the distal coupling has an inner diameter slightly smaller than that of the channel and the distal section of the proximal body of the bushing so that the first lateral projection becomes lodged against the distal coupling within the bushing.

In an embodiment, the core member includes a second failure point proximal of the first failure point, the core member defining the distal portion distal of the first failure point, a medial portion between the first and second failure points and the proximal section proximal of the second failure point.

The present disclosure also relates to a method for clipping tissue. The method includes inserting into a living body to a location adjacent to target tissue to be clipped a distal portion of a flexible elongate member while maintaining a proximal end of the flexible elongate member outside the living body, wherein a control member extends through an applicator and a bushing coupled to a distal end of the elongate flexible member, includes a proximal body coupled to a distal end of the flexible elongate member and a distal coupling movably attached to the proximal body; positioning a clip coupled to the bushing adjacent to the target tissue, the clip including a capsule releasably coupled to the distal coupling of the bushing via a first coupling member of the capsule hooked over a corresponding first hook of the distal coupling of the bushing, the capsule including a channel extending therethrough, and a pair of clip arms, proximal ends of which are slidably received within the channel; moving the control member distally relative to the elongate flexible member to move the clip arms to an open configuration, in which distal ends of the clip arms are separated from one another to receive tissue therebetween; drawing the control member proximally relative to the elongate flexible member to move the clip arms to a closed configuration, in which the distal ends of the clip arms are drawn toward one another so that the target tissue is gripped by the clip arms; and drawing the control member further proximally to increase a tension applied to a core member coupled to a distal end of the control member and received between and connected to the proximal ends of the clip arms until the tension reaches a predetermined level at which a distal portion of the core member is separated from a proximal portion thereof, wherein a lateral extent of a first lateral projection of the proximal portion of the core member being selected so that, when the distal portion of the core member is separated from the proximal portion of the core member, the first lateral projection is drawn proximally moving the distal coupling into the proximal body of the bushing driving the first coupling member radially outward beyond an outer end of the first hook to free the capsule from the bushing.

In an embodiment, the distal coupling is slidably coupled to the proximal body of the bushing for movement proximally and distally into and out of a distal end of the proximal body.

In an embodiment, an inner diameter of the channel is substantially equal to an inner diameter of a distal section of the proximal body of the bushing.

In an embodiment, the lateral extent of the first lateral projection is selected to be substantially equal to the inner diameters of the channel and the distal section of the proximal body of the bushing and wherein a proximal portion of the distal coupling has an inner diameter slightly smaller than that of the channel and the distal section of the proximal body of the bushing so that the first lateral projection becomes lodged against the distal coupling within the bushing.

In an embodiment, the core member includes a second failure point proximal of a first failure point, the core member defining the distal portion distal of the first failure point, a medial portion between the first and second failure points and a proximal section proximal of the second failure point.

### BRIEF DESCRIPTION OF THE DRAWINGS

FIG. 1 shows a side view of a distal portion of a clipping system according to an exemplary embodiment of the present disclosure with components of the clipping system separated from one another;

FIG. 2 shows a side view of the components connecting a control wire to a clip of the exemplary system of FIG. 1 with a capsule of the clip removed;

FIG. 3 shows a side view of a clip according to the embodiment of FIG. 1;

FIG. 4A shows a side view of a bushing according to the embodiment of FIG. 1 coupled to the capsule of a clip;

FIG. 4B shows a sideview of the bushing of FIG. 4A separated from the capsule of the clip;

FIG. 5A shows an exploded side view of the distal portion of the clipping system of FIG. 1;

FIG. 5B shows an exploded side view of the distal portion of the clipping system of FIG. 1 rotated 90 degrees relative to FIG. 5A;

FIG. 6 shows a partially cross-sectional view of the distal portion of the clipping system of FIG. 1;

FIG. 7A shows a bushing of the system of FIG. 1 with a movable part of the bushing separated from a proximal portion of the bushing;

FIG. 7B shows the bushing of FIG. 7A rotated 90 degrees relative to FIG. 7A;

FIG. 8 shows a partially cross-sectional view of a distal portion of the clipping system of FIG. 1 including a distal end of an applicator;

FIG. 9 shows a partially cross-sectional view of a distal portion of the clipping system of FIG. 1 including a distal end of the applicator a distal end of arms of the clip;

FIG. 10 shows a partially cross-sectional view of a distal portion of the clipping system of FIG. 1 including a distal end of the applicator with the capsule of the clip separated from the bushing and a distal portion of a core member severed from a medial portion of the core member;

FIG. 11 shows a partially cross-sectional view of a distal portion of the clipping system of FIG. 1 including a distal end of the applicator with the capsule of the clip separated from the bushing and a medial portion of a core member severed from a proximal portion of the core member;

FIG. 12A shows, in an open tissue receiving configuration, a partially cross-sectional view of the distal portion of the clipping system of FIG. 1 including a locking arrangement that locks the clip closed in a tissue clipping configuration;

FIG. 12B shows, in the closed tissue clipping configuration, a partially cross-sectional view of the distal portion of the clipping system of FIG. 1 including a locking arrangement that locks the clip closed in a tissue clipping configuration;

FIG. 13A shows a side view of a core member according to a further embodiment;

FIG. 13B shows a side view of the core member of FIG. 13A rotated 90 degrees relative to FIG. 13A;

FIG. 14 shows a partially cross-sectional view of a distal portion of a clipping system including a core member as shown in FIGS. 13A and 13B including a distal end of an applicator with a capsule of a clip separated from a bushing and a distal portion of a core member severed from a proximal portion of the core member; and

FIG. 15 shows a partially cross-sectional view of the clipping system of FIG. 14 including a handle, applicator and clip.

### DETAILED DESCRIPTION

The present disclosure may be further understood with reference to the following description and the appended drawings, wherein like elements are referred to with the same reference numerals. The present disclosure is directed to an endoscopic clipping system for treating internal tissue perforations, defects and/or bleeds. Exemplary embodiments of the present disclosure describe a clipping system comprising a clip releasably coupled to a proximal portion of the system, the clip including a pair of clip arms, proximal ends of which are slidable within a capsule to move the clip arms between an open configuration, in which distal ends of the clip arms are separated from one another to receive target tissue therebetween, and a closed configuration, in which the clip arms are drawn into the capsule so that the distal ends of the clip arms are moved toward one another to grip the target tissue therebetween. The clipping system includes a novel mechanism for separating the clip from an applicator minimizing the risk of shed and reducing the length of the clip.

The exemplary clipping system comprises an applicator including a flexible member extending from a handle to a distal end releasably coupled to a clip. The applicator includes a flexible elongate member extending from the handle with a control member slidably received therein. The control member is coupled to an actuator on the handle that permits a user to move the control member proximally and distally within the elongate member. A capsule of the clip is coupled to a distal end of the elongate member and the distal end of the control member is coupled to a unitary member that is slidable within the capsule and which couples the control member to arms of the clip. Movement of the control wire moves the unitary member proximally and distally so that the arms of the clip are extended from and drawn into the capsule to move the arms between an open tissue receiving configuration and a closed tissue clipping configuration.

A user deploys the clip by closing the clip arms over a target portion of tissue and applying proximal force to the unitary member via the control member until a failure point on the unitary member breaks. At this point, the clip arms are locked in the closed tissue clipping configuration and a part of the unitary member proximal of the failure point is drawn proximally out of the capsule separating the capsule from the elongate member.

As will be described in more detail below, the part of the unitary member proximal of the failure point includes projections extending radially outward therefrom configured to engage and bend radially outward portions of the proximal end of the capsule that had previously been bent over tabs at a distal end of the busing to releasably couple the bushing to the capsule. Thus, as the proximal portion of the unitary member is separated from the distal portion of the unitary member effectively separating the clip arms from the control member, and the capsule is separated from the bushing, the clip is completely separated from the applicator and the applicator can be removed from the body while leaving the clip clipped in place over the target tissue.

As will be described in more detail below, the construction of the unitary member and the bushing permit the housing of the severed proximal portion of the unitary member within the bushing. This permits the construction of a clip with a shorter capsule decreasing the total length of the clip as compared to the many clips available on the market such as the Resolution™ and Resolution 360™ clips available from Boston Scientific/SciMed. As will be understood by those of skill in the art, shorter clips may improve visualization of the target site and allow for better maneuverability when, for example, placing multiple clips. Some current clip designs also create shed parts during the deployment process of separating the clip from the catheter which shed parts may be left in the body to pass through the GI tract naturally. The embodiments described herein, minimize the risk that shed parts will be generated. Specifically, the control member is constructed to have a failure stress significantly higher than that of the unitary member. In addition, the severed proximal portion of the unitary member is, upon deployment, housed within the bushing and withdrawn from the body. Thus, the distal end of the control member is not severed from the proximal portion of the control member and the unitary member and distal end of the control member are controlled and prevented from becoming shed parts.

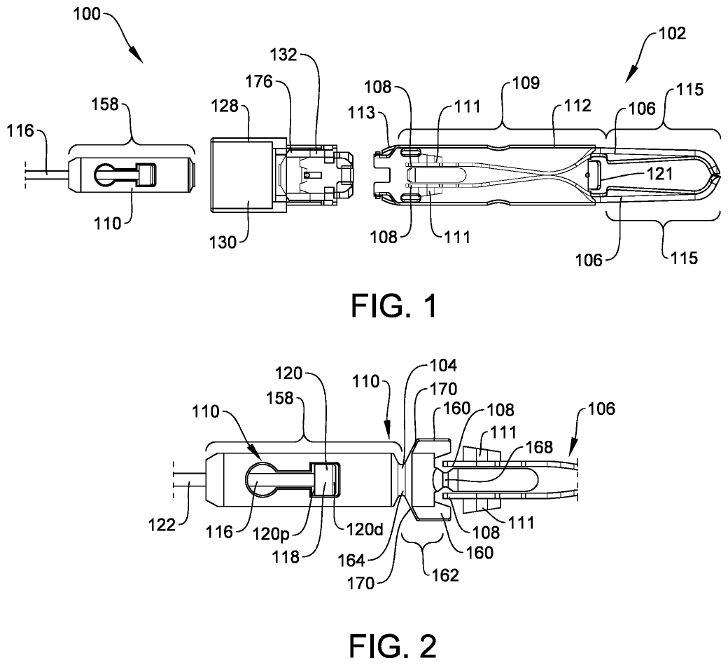

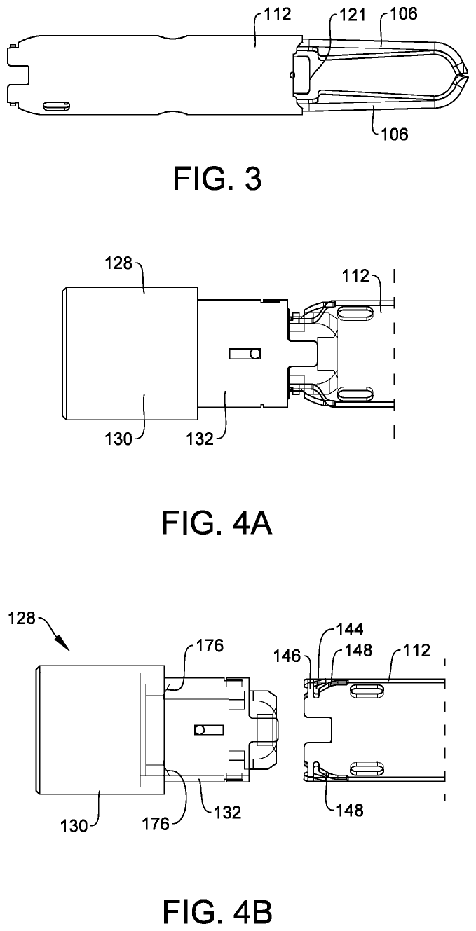

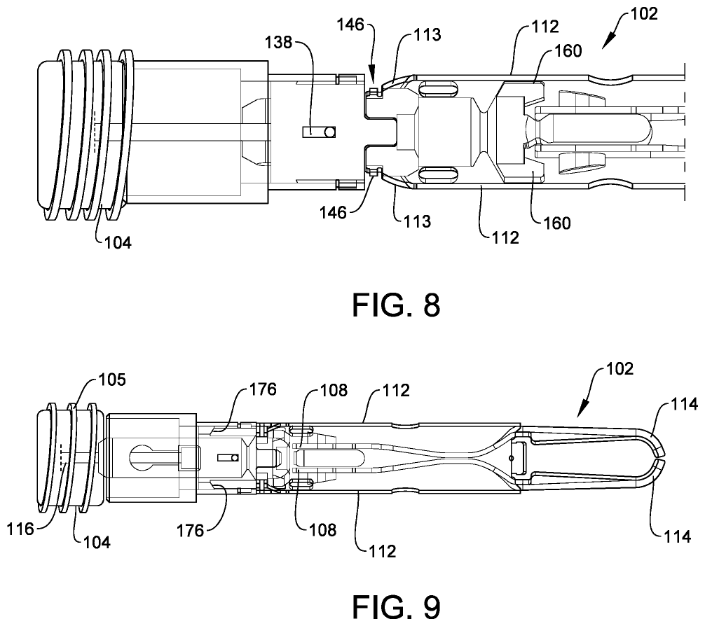

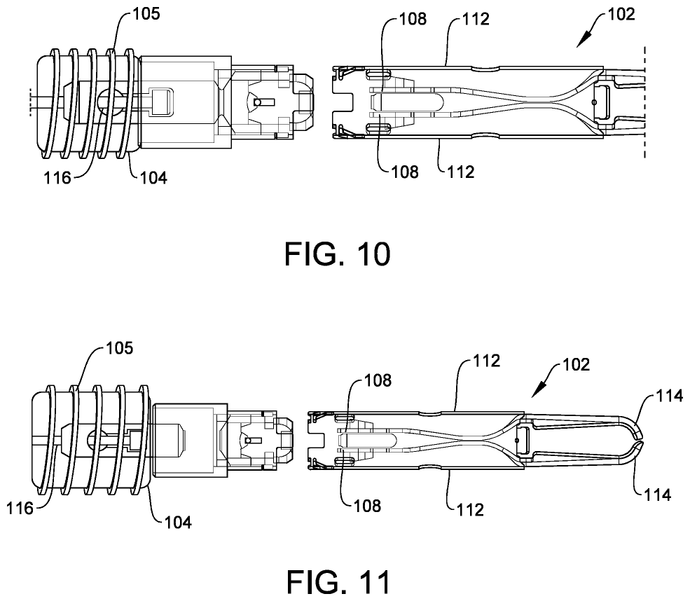

FIGS. 1-11 and 15 show a clipping system 100 for treating tissue defects comprising a clip 102 releasably coupled to an insertion device such as, for example, an applicator 104 (seen in FIGS. 8-11) (e.g., a catheter or other flexible tube). The clip 102 includes a pair of clip arms 106, proximal ends 108 of which are coupled to one another via a core member 110 which is slidably received within a capsule 112 to move the clip 102 between an open configuration, in which distal ends 114 of the clip arms 106 are separated from one another to receive a tissue therebetween, and a closed configuration, in which the distal ends 114 of the clip arms 106 are drawn toward one another to grip tissue therebetween.

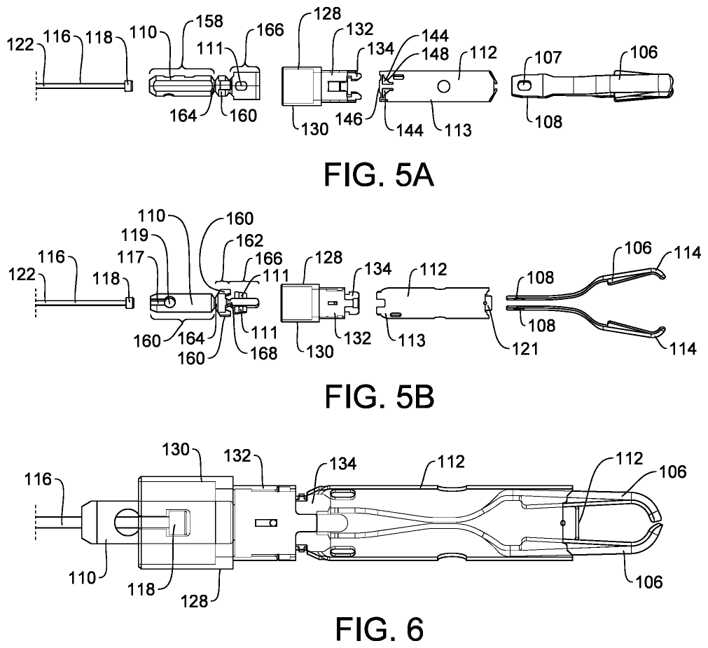

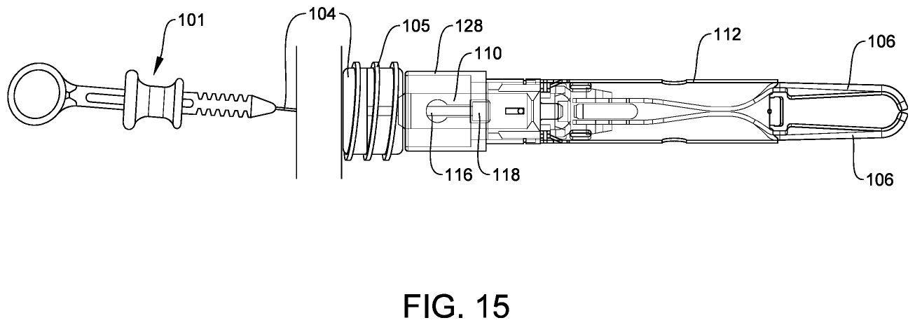

The clip arms 106 are moved between the open and closed configurations via a control member 116, a distal end 118 of which is received within a cavity 120 of the core member 110 while a reduced dimension proximal portion 122 of the control member 116 extends proximally therefrom to pass out of the core member 110 (see FIG. 2). As shown in FIG. 15, the proximal portion 122 of the control member 116 extends through an applicator 104 to a proximal end that is accessible to a user of the system 100. In this embodiment, the distal end 118 of the control member is enlarged (i.e., of increased diameter) relative to the proximal portion 122 of the control member 116. As can be seen in FIG. 5B, the core member 110 includes an opening 119 sized and shaped to permit the distal end 118 to be passed therethrough.

The control member 116 is then rotated to seat the distal end 118 in the cavity 120 which is sized and shaped to snugly receive the distal end 118. In this embodiment, the distal end 118 and the cavity 120 are both substantially cylindrical with the cavity 120 slightly larger than the distal end 118 by tolerances that permit the distal end 118 to be securely seated therein. As can be seen in FIG. 5B, a longitudinal indentation 117 is formed on a side of the core member 110 opposite of the side within which the cavity 120 is formed and extends proximally from the opening 119 so that, when the distal end 118 is rotated into position within the cavity 120, the distal-most part of the proximal portion 122 of the control member 116 is seated in the longitudinal indentation 117. Thus, when the user moves the control member 116 longitudinally (i.e., proximally and distally) relative to the applicator 104, the distal end 118 engages proximal and distal ends, 120p and 120d, respectively, of the cavity 120 so that the core member 110 and the clip arms 106 are moved proximally and distally relative to the capsule 112 so that the clip 102 moves between the open and the closed configurations.

In particular, movement of the control member 116 distally relative to the capsule 112 moves the clip arms 106 out of the capsule 112 so that the clip arms 106 spring apart from one another (e.g., under a natural bias imparted to the clip arms 106). In one embodiment, each of the clip arms 106 is formed of an elastic material such as nitinol and is pre-shaped (e.g., heat formed) to a shape matching a shape of the clip arm 106 when in the open configuration. Thus, when not constrained by the capsule 112 to remain in the closed configuration, the clip arms 106 spring apart from one another into the open configuration.

As will be explained in more detail below, the clip 102 is releasably coupled to the applicator 104 via a bushing 128 coupled to a distal end of the applicator 104 so that the applicator 104 and the bushing 128 form an applicator that is inserted through an insertion instrument (e.g., a flexible endoscope) to a target site within a living body. The part of the applicator 104 extending proximally from the bushing 128 to a handle 101 (See, FIG. 15) is formed, in this embodiment, as a coil 105 of a flexible material such as, for example, stainless steel, so that the applicator 104 can navigate even the tortuous paths traversed by the most flexible endoscopes.

A desired orientation of the clip arms 106 within the capsule 112 is maintained, in this embodiment, by a tab 121 that is formed as a portion of the capsule 112 folded radially inward across a distal opening of the capsule 112 and received between the clip arms 106. The size of the tab 121 is selected so that the space allowed to each clip arm 106 in the open distal end of the capsule 112 is sufficient to permit the clip arms 106 to slide into and out of the capsule 112 only when aligned with the tab 121. The proximal end 108 of each clip arm 106 is coupled to a distal end of the core member 110 via pins 111 extending radially outward from opposite sides of the core member 110.

Each of the pins 111 passes through an opening 107 in the proximal end of a corresponding one of the clip arms 106 so that the clip arms 106 so that the clip arms 106 are moved proximally and distally within the capsule 112 when the control member 116 is moved proximally and distally through the applicator 104. Thus, movement of the control member 116 relative to the applicator 104 moves the clip arms 106 proximally into and distally out of the capsule 112 so that the clip arms 106 move between the open and closed configurations. In addition, as the clip arms 106 are held in a desired orientation within the capsule 112 by the tab 121, the connection between the pins 111 and the clip arms 106 maintains a desired orientation of the core member 110 within the capsule 112.

As described above, the applicator 104 includes a bushing 128 at a distal end thereof for releasably coupling the applicator 104 to the clip 102. The control member 116 extends through the applicator 104 distally into the bushing 128 where it connects to the core member 110 as described above. As seen in FIGS. 5A and 5B, the bushing 128 of this embodiment includes a proximal section 130 having an outer diameter equal to the outer diameter of the distal end of the coil 105 and a distal section 132 having an outer diameter reduced with respect to the proximal section 130. The outer diameter of the distal section 132 in this embodiment is equal to the outer diameter of the capsule 112.

As would be understood by those skilled in the art, the coil 105, the bushing 128 and the capsule 112 of this embodiment are all generally cylindrical. However, the shape of these components can be varied in any manner desired so long as the applicator and the clip 102 may be inserted through a working channel of a delivery device (e.g., flexible endoscope) and manipulated as desired without damaging the delivery device. The bushing 128 in this device operates to ensure that the clip 102 is completely separated from the applicator 104 after the user has deployed the clip 102 while also eliminating shed parts. Specifically, it is important that the applicator 104 be completely separated from the clip 102 after deployment to prevent a situation in which the clip 102 is locked and gripped over tissue in the body while the applicator remains connected to the clip. As would be understood by those skilled in the art, such failure to separate the applicator 104 from the clip 102 may require a separate procedure to separate the clip from either the tissue over which it is clipped or from the applicator.

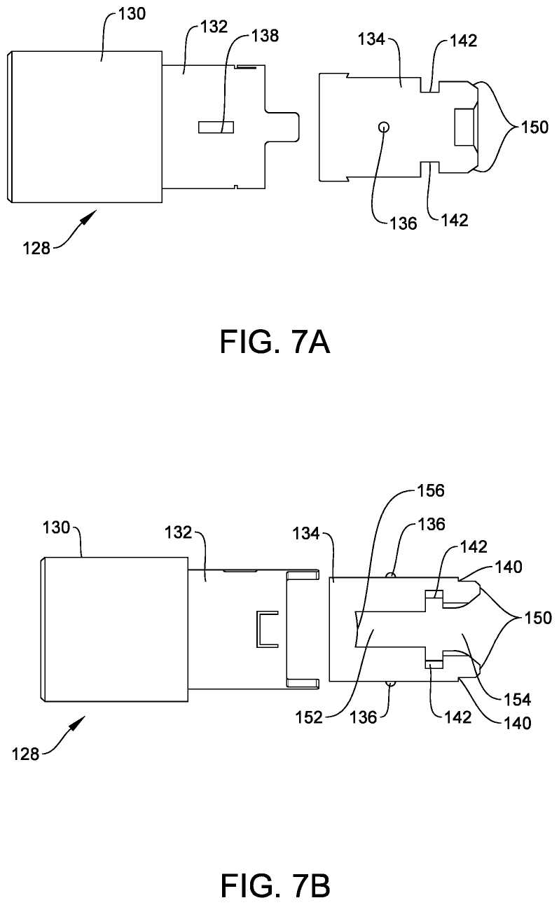

In this embodiment, the bushing 128 includes a moving coupler 134 that is slidably received within the distal section 132. A pair of pins 136 extending radially outward from opposite sides of the moving coupler 134 of this embodiment are received in corresponding slots 138 extending longitudinally through a portion of each of the opposite sides of the distal section 132 so that the moving coupler 134 may slide proximally and distally within the distal section 132 along a distance defined by the slots 138. The moving coupler 134 includes a pair of distally extending wings 140 each of which defines two niches 142 (See, FIGS. 7A and 7B). Each of the niches is sized and positioned to receive a corresponding locking feature at a proximal end 113 of the capsule 112.

In this embodiment, the locking features of the capsule are formed as a series of openings 144 formed between a proximal web 146 and a proximal end 148 of a cylindrical part of the capsule 112. Before assembly, the proximal end of the capsule 112 is substantially cylindrical and, when the moving coupler 134 is inserted into the capsule 112, the proximal end of the capsule is compressed radially inward so that each of the webs 146 is forced into a corresponding one of the niches 142 with each of a plurality of tabs 150 (in this embodiment 4 tabs 150) of the moving coupler 134 that extend distally of the niches 142 is received within a corresponding one of the openings 144 releasably locking the capsule 112 to the bushing 128. As can be seen in FIGS. 5A and 7B, the wings 140 of the moving coupler 134 are separated from one another via slots 152, each of which extends longitudinally from an open distal end 154 to a closed proximal end 156. In this configuration, the webs 146 extend across the slots 152.

The core member 110 of this embodiment includes a proximal portion 158 coupled to a medial portion 162 extending distally from the proximal portion 158 and coupled thereto by a first fracture point 164. A distal portion 166 extending distally from the medial portion 162 is coupled to the medial portion 162 via a second fracture point 168. The proximal portion 158 includes the opening 119 and the cavity 120 as well as the longitudinal indentation 117, while the medial portion 162 includes a pair of diametrically opposed lateral projections 160 that extend outward by a distance selected to substantially match an inner diameter of the capsule 112 as well as an inner diameter of the distal section 132 of the bushing 128. The distal portion 166 includes the pins 111 and two opposed surfaces 268 on which the proximal ends 108 of the clip arms 106 are received.

As described below, each of the lateral projections 160 is moveable to contact and bend radially outward a corresponding part of the capsule 112 that had been hooked over a distal part of the bushing 128. When drawn proximally out of the capsule 112, the lateral projections 160 bend these parts of the capsule 112 radially outward beyond an extent of parts of the bushing 128 over which they had been hooked to free the capsule 112 from the bushing 128. Thus, the lateral projections 160 should preferably extend radially outward by a distance greater than a radial extent of the parts of the bushing 128 over which the capsule 112 is hooked so that these parts of the capsule are pushed radially outward beyond this part of the bushing 128. As described below, it may be desired to construct the lateral projections 160 so that they extend radially out to slide on an inner surface of the capsule 112 as this will allow for the maximum radially outward expansion of the hooked parts of the capsule 112.

The core member 110 and the bushing 128 of this embodiment operate together to ensure that, upon deployment of the clip 102 to lock over target tissue, the clip 102 will be successfully separated from the applicator 104 so that the applicator 104 may be withdrawn from the body while leaving the clip 102 in place clipped over the target tissue. Specifically, it is noted that when the clip 102 is assembled the clip arms 106 and, consequently, the core member 110 are maintained in a desired orientation within the capsule 112 via the tab 121. In this desired orientation, the medial and distal portions 162, 166, respectively, of the core member 110 are received within the capsule 112 with the lateral projections 160 aligned with the slots 152 in the distal section 132 of the bushing 128.

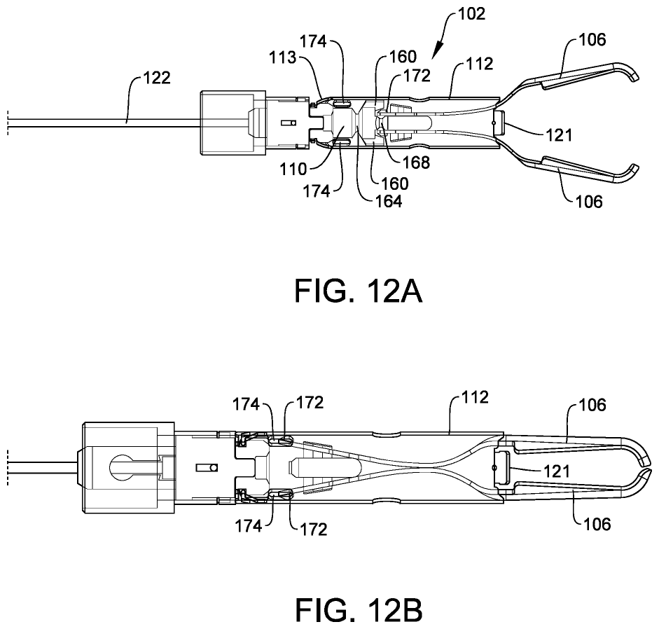

As shown in FIGS. 12A and 12B, each of the clip arms 106 of the clip 102 of this embodiment includes a locking feature at a proximal end 108 thereof. In this embodiment, the locking feature of each clip arm 106 is formed as a tab 172 and the proximal ends 108 of the clip arms 106 are biased to spring radially outward. The proximal ends 108 of the clip arms 106 are constrained to a radially inner position in which the tabs 172 are separated from the capsule 112 by overhangs of the lateral projections 160. Thus, while the clip 102 is moved between the open and closed configurations and the core member 110 remains intact, the lateral projections 160 maintain the proximal ends 108 and the tabs 172 in an unlocked configuration so that the clip 102 may be opened and closed until a desired portion of tissue has been gripped by the clip 102. When the user determines that a target portion of tissue has been gripped as desired and the clip 102 is to be deployed, as will be described in more detail below, the user operates an actuator on the handle 101 until the tension on the control member 116 and the core member 110 exceeds a predetermined level at which the core member 110 fails at the second fracture point 168 so that proximal and medial portions 158, 162, respectively, of the core member 110 including the lateral projections 160 are moved proximally relative to the clip 102. Thus, when the lateral projections 160 no longer overhang the proximal ends 108 of the clip arms 106, the tabs 172 are freed to spring radially outward.

The tabs 172 are positioned so that, when the clip arms 106 have been drawn proximally into the capsule 112 to a maximum extent and the lateral projections 160 are withdrawn from the capsule 112, the tabs 172 spring outward to enter windows 174 formed adjacent to the proximal end 113 of the capsule. Each of the tabs 172 engages a distal surface of a corresponding one of the windows 174 so that the clip arms 106 are locked in the closed configuration gripping the target tissue. Those skilled in the art will understand that this locking mechanism is exemplary only and that any of a variety of known clip locking mechanisms may be substituted for this exemplary mechanism. The proximal and medial portions 158, 162, respectively, of the core member 110 are drawn into the bushing 128 and the distal portion 166 of the core member 110 is retained within the capsule 112 so that deployment of the clip 102 does not result in any shed parts.

As would be understood by those skilled in the art, in use, the user advances the clip 102 to a target site adjacent to target tissue to be clipped (e.g., by advancing the applicator 104 and the clip 102 through the working channel of a flexible endoscope). When the clip 102 is positioned as desired, the user advances the control member 116 distally relative to the applicator 104 (e.g., via the thumb ring and slider of the handle 101) to move the core member 110 and the clip arms 106 distally through the capsule 112. As the clip arms 106 project distally out of the capsule 112, the clip arms 106 spread apart from one another into the open tissue receiving configuration under their natural bias. The clip 102 is then further advanced distally until the distal ends 114 of the clip arms 106 contact the target tissue.

While maintaining distally directed pressure against the target tissue, the user withdraws the control member 116 proximally so that the clip arms 106 are drawn together as they are withdrawn into the capsule 112. This causes the distal ends 114 of the clip arms to grip the tissue positioned between the clip arms 106 until the clip arms 106 reach a point beyond which they can be drawn no further proximally into the capsule. For example, the clip arms 106 of this embodiment include distal portions 115 that are wider than the proximal portions 109 of the clip arms 106. The narrower proximal portions 109 of the clip arms 106 have a width that permits them to be received within the capsule 112 while the width of the distal portions 115 is larger than can be accommodated within the capsule 112. Thus, when the user has withdrawn the clip arms 106 proximally into the capsule 112 to the point where proximal ends of the distal portions 115 of the clip arms 106 contact the distal end of the capsule 112, continued proximally directed force applied to the control member 116 increases a tension on the control member 116 and, consequently, on the core member 110.

When this tension reaches a first threshold level, the second fracture point 168 fractures separating the distal portion 166 from the medial portion of the core member 110. This frees the medial and proximal portions 162, 158, respectively, of the core member 110 to move proximally. As the medial portion 162 moves proximally through the open proximal end of the capsule 112, the lateral projections 160 move proximally through the slots 152 so that ramped proximal surfaces 170 of the lateral projections engage the webs 146 and bend them radially outward out of the niches 142. When the lateral projections 160 contact the closed ends 156 of the slots 152, further proximal motion of the medial portion 162 and the lateral projections 160 drives the moving coupler 134 proximally into the distal section 132.

As the lateral projections 160 are drawn into the distal section, the webs 146 will be forced radially outward ensuring that they are driven out of the niches 142 separating the bushing 128 from the capsule 112. The lateral projections 160 move proximally into the distal section 132 until they contact a hard stop 176 extending radially inward within the distal section 132 of the bushing 128 that is configured to define a proximal-most point to which the lateral projections and the medial portion 162 of the core member 110 may be drawn into the bushing 128.

Additional proximally directed tension after this point has been reach thus increases tension applied to the control member 116 and the proximal and medial portions 158, 162, respectively, of the core member 110. When a second threshold tension (a tension greater than the first threshold tension) is reached, the first fracture point 164 fails and the medial portion 162 is separated from the proximal portion 158.

Those skilled in the art will understand that the embodiments described herein reduce the risk of shed parts by designing the control member 116 to have strength sufficient to remain intact when the tension(s) required to sever the failure point(s) of the core member 110 are reached so that the control member 116 will not fail during deployment of the clip. That is, the core member 110 will fail before a level of tension on the control member 116 reaches a level at which the control member 116 would fail. The distal end of the control member 116 remains coupled to the proximal portion of the control member 116 even after the clip has been deployed and is retained within the proximal portion of the core member 110 within the bushing 128 for removal from the body. As only the distal portion 166 of the core member 110 remains within the capsule 1112 and the medial and proximal portions 162, 158, respectively, of the core member 110 are withdrawn proximally into the bushing 128 for withdrawal from the body, the clip 102 can be made shorter than most conventional clips. For example, clips according to the disclosed embodiments can be as short as 8 mm to 9 mm in length (from distal ends 114 of the clip arms 106 to a proximal end of the capsule 112).

Those skilled in the art will understand that this operation provides multiple instances of feedback to the user. First, the user will understand that, after opening the clip 102 and closing it over target tissue, the increase in resistance to further proximal movement of the control member 116 indicates that the distal portions 115 of the clip arms 106 have contacted the distal end of the capsule 112. Then, as increasing tension is applied to the control member 116, the user will feel a loosening of the tension (and may hear a click) when the second fracture point 168 fails. This lets the user know that the clip 102 has been locked clipped over target tissue.

Then, after further proximal movement of the control member 116, the user will feel the new resistance to further proximal movement of the control member 116 when the medial portion 162 becomes lodged in the distal section 132 of the bushing 128. Finally, after increasing the tension on the control member, the user will feel the loosening of the tension on the control member 116 and may hear another click as the first fracture point 164 fails. This will inform the user that the clip 102 has been successfully clipped over target tissue and that the clip 102 has been separated from the applicator 104. The applicator can then be withdrawn from the insertion instrument leaving the clip 102 in place within the body.

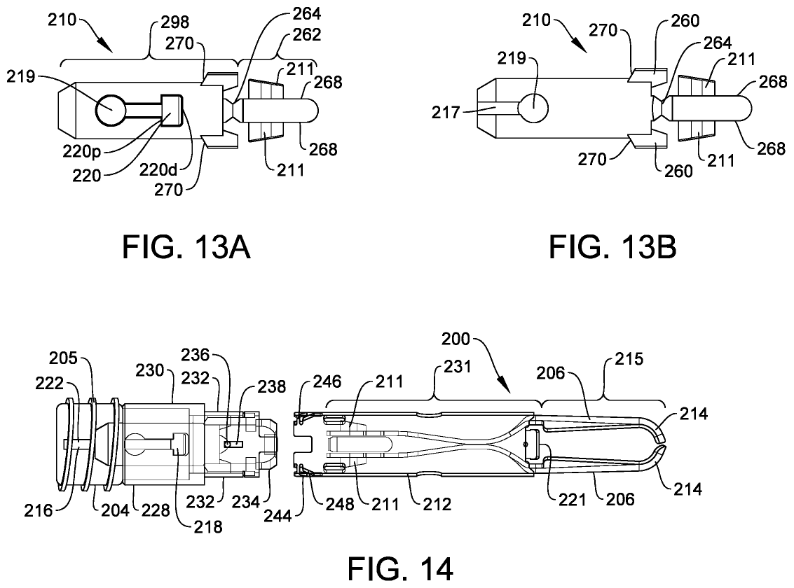

FIGS. 13A, 13B and 13, show a clipping system 200 according to a further embodiment comprising a clip 202 releasably coupled to an insertion device such as, for example, an applicator 204. The clip 202 includes a pair of clip arms 206, proximal ends 208 of which are coupled to one another via a core member 210 which is slidably received within a capsule 212 to move the clip 202 between an open configuration, in which distal ends 214 of the clip arms 206 are separated from one another to receive a tissue therebetween, and a closed configuration, in which the distal ends 214 of the clip arms 206 are drawn toward one another to grip tissue.

The clip arms 206 are moved between the open and closed configurations via a control member 216, a distal end 218 of which is received within a cavity 220 of the core member 210 while a reduced dimension proximal portion 222 of the control member 216 extends proximally therefrom to pass out of the core member 210. The proximal portion 222 of the control member 216 extends through an applicator 204, to a proximal end that is accessible to a user of the system 200. In this embodiment, the distal end 218 of the control member is enlarged (i.e., of increased diameter) relative to the proximal portion 222 of the control member 216. The core member 210 includes an opening 219 sized and shaped to permit the distal end 218 to be passed therethrough.

As described above in regard to the system 100, the control member 216 is then rotated to seat the distal end 218 in the cavity 220 which is sized and shaped to snugly receive the distal end 218. In this embodiment, the distal end 218 and the cavity 220 are both substantially cylindrical with the cavity 220 slightly larger than the distal end 218 by tolerances that permit the distal end 218 to be securely seated therein. As can be seen in FIG. 13B, a longitudinal indentation 217 is formed on a side of the core member 210 opposite of the side within which the cavity 220 is formed and extends proximally from the opening 219 so that, when the distal end 218 is rotated into position within the cavity 220, the distal-most part of the proximal portion 222 of the control member 216 is seated in the longitudinal indentation 217. Thus, when the user moves the control member 216 longitudinally (i.e., proximally and distally) relative to the applicator 204, the distal end 218 engages the proximal and distal ends, 220p and 220d, respectively, of the cavity 220 so that the core member 210 and the clip arms 206 are moved proximally and distally relative to the capsule 212 so that the clip 202 moves between the open and the closed configurations.

Movement of the control member 216 distally relative to the capsule 212 moves the clip arms 206 out of the capsule 212 so that the clip arms 206 spring apart from one another (e.g., under a natural bias imparted to the clip arms 206). In one embodiment, each of the clip arms 206 is formed of an elastic material such as nitinol and is pre-shaped (e.g., heat formed) to a shape matching a shape of the clip arm 206 when in the open configuration. Thus, when not constrained by the capsule 212 to remain in the closed configuration, the clip arms 206 spring apart from one another into the open configuration.

As will be explained in more detail below, the clip 202 is releasably coupled to the applicator 204 via a bushing 228 coupled to a distal end of the applicator 204 so that the applicator 204 and the bushing 228 form an applicator that is inserted through an insertion instrument (e.g., a flexible endoscope) to a target site within a living body. The part of the applicator 204 extending proximally from the bushing 228 to a handle as shown, e.g., in FIG. 15, is formed as a coil 205 of flexible material such as, for example, stainless steel, permitting the applicator 204 to navigate tortuous paths traversed by the most flexible endoscopes.

A desired orientation of the clip arms 206 within the capsule 212 is maintained, in this embodiment, by a tab 221 that is folded radially inward across a distal opening of the capsule 212 and received between the clip arms 206. The size of the tab 221 is selected so that the space allowed to each clip arm 206 in the open distal end of the capsule 212 is sufficient to permit the clip arms 206 to slide into and out of the capsule 212 only when aligned with the tab 221. The proximal end 208 of each clip arm 206 is coupled to a distal end of the core member 210 via pins 211 extending radially outward from opposite sides of the core member 210. Each of the pins 211 passes through an opening 207 in the proximal end of a corresponding one of the clip arms 206 so that the clip arms 206 so that the clip arms 206 are moved proximally and distally within the capsule 212 when the control member 216 is moved proximally and distally through the applicator 204. Movement of the control member 216 relative to the applicator 204 moves the clip arms 206 proximally into and distally out of the capsule 212 so the clip arms 206 move between the open and closed configurations. In addition, as the clip arms 206 are held in a desired orientation within the capsule 212 by the tab 221, the connection between the pins 211 and the clip arms 206 maintains a desired orientation of the core member 210 within the capsule 212.

As described above, the applicator 204 includes the bushing 228 at a distal end thereof for releasably coupling the applicator 204 to the clip 202. The bushing 228 and its coupling to the capsule 212 is substantially identical to that described in regard to the bushing 128 and the capsule 112 and the interaction between the core member 210 and the connection between the bushing 228 and the core member 210 is also substantially the same as that described above in regard to the operation of the core member and its effect in separating the capsule 112 from the bushing 1128. As seen in FIG. 14, the bushing 228 includes a proximal section 230 having an outer diameter equal to the outer diameter of the distal end of the coil 205 and a distal section 232 having an outer diameter reduced with respect to the proximal section 230. The outer diameter of the distal section 232 is equal to the outer diameter of the capsule 212. The bushing 228 includes a moving coupler 234 slidably received within the distal section 232.

A pair of pins 236 extending radially outward from opposite sides of the moving coupler 234 are received in corresponding slots 238 extending longitudinally through a portion of each of the opposite sides of the distal section 232 so that the moving coupler 234 may slide proximally and distally within the distal section 232 along a distance defined by the slots 238. The moving coupler 234 includes a pair of distally extending wings 240 each of which defines two niches 242. Each of the niches 242 is sized and positioned to receive a corresponding locking feature at a proximal end 213 of the capsule 212. In this embodiment, the locking features of the capsule are formed as a series of openings 244 formed between a proximal web 246 and a proximal end 248 of a cylindrical part of the capsule 212.

As seen in FIG. 14, before assembly, the proximal end of the capsule 212 is substantially cylindrical and, when the moving coupler 234 is inserted into the capsule 212, the proximal end of the capsule 212 is compressed radially inward so that each of the webs 246 is forced into a corresponding one of the niches 242 with each of a plurality of tabs 250 (in this embodiment 4 tabs 250) of the moving coupler 234 that extend distally of the niches 242 is received within a corresponding one of the openings 244 releasably locking the capsule 212 to the bushing 228. The wings 240 of the moving coupler 234 are separated from one another via slots similar to the slots 152 of the bushing 128 each of which extends longitudinally from an open distal end to a closed proximal end. In this configuration, the webs 246 extend across the slots 252 in the same manner described above in regard to the system 100.

The core member 210 of this embodiment includes a proximal portion 258 coupled to a distal portion 262 extending distally from the proximal portion 258 and coupled thereto by a fracture point 264. The proximal portion 258 includes the opening 219 and the cavity 120 as well as the longitudinal indentation 217 while the distal portion 262 includes a pair of diametrically opposed lateral projections 260 that extend outward by a distance selected to substantially match an inner diameter of the capsule 212 as well as an inner diameter of the distal section 232 of the bushing 228. The distal portion 262 also includes the pins 211 and two opposed surfaces 268 on which the proximal ends 208 of the clip arms 206 are received.

The core member 210 and the bushing 228 of this embodiment operate together in a manner substantially similar to that of the core member 110 and the bushing 128 to ensure that, upon deployment of the clip 202 to lock over target tissue, the clip 202 will be successfully separated from the applicator 204 so that the applicator 204 may be withdrawn from the body while leaving the clip 202 in place clipped over the target tissue. Specifically, it is noted that when the clip 202 is assembled the clip arms 206 and, consequently, the core member 210 are maintained in a desired orientation within the capsule 212 via the tab 221. In this desired orientation, a distal end of the proximal portion 258 including the lateral projections 260 and the distal portion 262 of the core member 210 are received within the capsule 212 with the lateral projections 260 aligned with the slots in the distal section 232 of the bushing 228.

As would be understood by those skilled in the art, in use, the user advances the clip 202 to a target site adjacent to target tissue to be clipped (e.g., by advancing the applicator 204 and the clip 202 through the working channel of a flexible endoscope). When the clip 202 is positioned as desired, the user advances the control member 216 distally relative to the applicator 204 to move the core member 210 and the clip arms 206 distally through the capsule 212. As the clip arms 206 project distally out of the capsule 212, the clip arms 206 spread apart from one another into the open tissue receiving configuration under their natural bias. The clip 202 is then further advanced distally until the distal ends 214 of the clip arms 206 contact the target tissue.

While maintaining distally directed pressure against the target tissue, the user withdraws the control member 216 proximally so that the clip arms 206 are drawn together as they are withdrawn into the capsule 212. This draws the distal ends 214 of the clip arms 206 together gripping tissue positioned between the clip arms 206 until the clip arms 206 reach a point beyond which they can be drawn no further proximally into the capsule 212. The clip arms 206 of this embodiment include distal portions 215 that are wider than proximal portions 231 of the clip arms 206.

The narrower proximal portions 231 of the clip arms 206 have a width that permits them to be received within the capsule 212 while the width of the distal portions 215 is larger than can be accommodated within the capsule 212. Thus, when the user has withdrawn the clip arms 206 proximally into the capsule 212 to the point where proximal ends of the distal portions 215 of the clip arms 206 contact the distal end of the capsule 212, continued proximally directed force applied to the control member 216 increases a tension on the control member 216 and, consequently, on the core member 210.

When this tension reaches a threshold level, the fracture point 264 fractures separating the distal portion 166 from the proximal portion of the core member 210. This frees the proximal portion 158 of the core member 210 to move proximally. As the distal end of the proximal portion 158 moves proximally through the open proximal end of the capsule 212, the lateral projections 260 move proximally through the slots in the distal portion of the distal section 232 of the bushing 228 so that ramped proximal surfaces 270 of the lateral projections 260 engage the webs 246 and bend them radially outward out of the niches 242.

When the lateral projections 260 contact the closed ends of the slots in the distal section 232, further proximal motion of the proximal portion 158 and the lateral projections 260 drives the moving coupler 234 proximally into the distal section 232. As the lateral projections 260 are drawn into the distal section 232 whose inner diameter substantially matches the radial extent of the lateral projections 260, the webs 246 will be forced radially outward along the ramped surfaces 270 ensuring that they are driven out of the niches 242 separating the bushing 228 from the capsule 212. The lateral projections 260 then become lodged within the distal section 232 through contact with the inner wall thereof so that further proximal motion of the proximal portion 158 is prevented.

Those skilled in the art will understand that this operation provides multiple instances of feedback to the user. First, the user will understand that, after opening the clip 202 and closing it over target tissue, the increase in resistance to further proximal movement of the control member 216 indicates that the distal portions 215 of the clip arms 206 have contacted the distal end of the capsule 212. Then, as increasing tension is applied to the control member 216, the user will feel a loosening of the tension (and may hear a click) when the fracture point 264 fails. This lets the user know that the clip 202 has been locked clipped over target tissue. Then, after further proximal movement of the control member 216, the user will feel the new resistance to further proximal movement of the control member 216 when the proximal portion 158 becomes lodged in the distal section 232 of the bushing 228. This will inform the user that the clip 202 has been successfully clipped over target tissue and that the clip 202 has been separated from the applicator 204. The applicator can then be withdrawn from the insertion instrument leaving the clip 202 in place within the body.

It will be apparent to those skilled in the art that various modifications may be made without departing from the scope of the disclosure. Although specific embodiments have been illustrated and described herein, it should be appreciated that any arrangement calculated to achieve the same purpose may be substituted for the specific embodiments shown. This disclosure is intended to cover any and all adaptations or variations of various embodiments. It is to be understood that the above description has been made in an illustrative fashion, and not a restrictive one. Combinations of the above embodiments, and other embodiments not specifically described herein will be apparent to those of skill in the art upon reviewing the above description. Thus, the scope of various embodiments includes any other applications in which the above compositions, structures, and methods are used.

1-15. (canceled)

16. A clipping system for treating tissue, comprising:
an applicator including a flexible elongate member extending from a proximal end which, during use, remains outside a living body to a distal end which, during use, is inserted into the living body to a location adjacent to target tissue to be clipped, the applicator including:
a control member extending through the applicator; and
a bushing coupled to a distal end of the elongate flexible member, the bushing including a proximal body coupled to a distal end of the flexible elongate member and a distal coupling movably attached to the proximal body;
a clip including a capsule releasably coupled to the distal coupling of the bushing via a first coupling member of the capsule hooked over a corresponding first hook of the distal coupling of the bushing, the capsule including a channel extending therethrough, and a pair of clip arms, proximal ends of which are slidably received within the channel for movement between an open configuration, in which distal ends of the clip arms are separated from one another to receive tissue therebetween, and a closed configuration, in which the distal ends of the clip arms are drawn toward one another to grip tissue; and
a core member received between and connected to the proximal ends of the clip arms and including a first lateral projection and a first failure point distal of the first lateral projection, the first failure point separating a distal portion of the core member from a proximal portion thereof, the core member being coupled to a distal end of the control member so that longitudinal movement of the control member relative to the elongate flexible member moves the clip arms between the open configuration and the closed configuration, a lateral extent of the first lateral projection being selected so that, when the clip is clipped over target tissue, the first lateral projection is drawn proximally moving the distal coupling into the proximal body of the bushing, the first lateral projection driving the first coupling member radially outward beyond an outer end of the first hook to free the capsule from the bushing.

17. The system of claim 16, wherein the core member includes a first pin received within a hole in a proximal part of a first one of the clip arms and a second pin received within a hole in a proximal part of a second one of the clip arms.

18. The system of claim 16, wherein the distal coupling is slidably coupled to the proximal body of the bushing for movement proximally and distally into and out of a distal end of the proximal body.

19. The system of claim 16, wherein the proximal body of the bushing includes a proximal section having an outer diameter substantially equal to an outer diameter of the flexible elongate member and a distal section of the proximal body of the bushing has an outer diameter substantially equal to an outer diameter of the capsule.

20. The system of claim 19, wherein an inner diameter of the channel is substantially equal to an inner diameter of a distal section of the proximal body of the bushing.

21. The system of claim 20, wherein the lateral extent of the first lateral projection is selected to be substantially equal to the inner diameters of the channel and the distal section of the proximal body of the bushing and wherein a proximal portion of the distal coupling has an inner diameter slightly smaller than that of the channel and the distal section of the proximal body of the bushing so that the first lateral projection becomes lodged against the distal coupling within the bushing.

22. The system of claim 21, wherein the core member includes a second failure point proximal of the first failure point, the core member defining the distal portion distal of the first failure point, a medial portion between the first and second failure points and the proximal section proximal of the second failure point.

23. The system of claim 22, wherein the clip arms are constructed to define a proximal-most position of the clip arms within the capsule so that, when a proximally tension applied to the control member after the clip arms have reached the proximal-most position exceeds a first threshold level, the core member separates at the first failure point permitting the medial and proximal portions of the core member to move proximally drawing the first lateral projection proximally out of the capsule driving the first coupling member of the capsule radially outward beyond an outer end of the first hook of the distal coupling of the bushing to separate the capsule from the bushing.

24. A device for clipping tissue, comprising:
a bushing including a proximal body configured to be coupled to a distal end of a flexible applicator and a distal coupling movably attached to the proximal body, the distal coupling including a first hook;
a clip including a capsule releasably coupled to the distal coupling via a first coupling member of the capsule hooked over the first hook, the capsule including a channel extending therethrough, and a pair of clip arms, proximal ends of which are slidably received within the channel for movement between an open configuration, in which distal ends of the clip arms are separated from one another to receive tissue therebetween, and a closed configuration, in which the distal ends of the clip arms are drawn toward one another to grip tissue; and
a core member received between and connected to the proximal ends of the clip arms and including a first lateral projection and a first failure point distal of the first lateral projection, the first failure point separating a distal portion of the core member from a proximal portion thereof, the core member configured to be coupled to a distal end of a control member so that longitudinal movement of the control member relative to the flexible applicator moves the clip arms between the open configuration and the closed configuration, a lateral extent of the first lateral projection being selected so that, when the clip is clipped over target tissue, the first lateral projection is drawn proximally moving the distal coupling into the proximal body of the bushing, the first lateral projection driving the first coupling member radially outward beyond an outer end of the first hook to free the capsule from the bushing.

25. The device of claim 24, wherein the core member includes a first pin received within a hole in a proximal part of a first one of the clip arms and a second pin received within a hole in a proximal part of a second one of the clip arms.

26. The device of claim 24, wherein the distal coupling is slidably coupled to the proximal body of the bushing for movement proximally and distally into and out of a distal end of the proximal body.

27. The device of claim 24, wherein the proximal body of the bushing includes a proximal section having an outer diameter substantially equal to an outer diameter of the flexible applicator and a distal section of the proximal body of the bushing has an outer diameter substantially equal to an outer diameter of the capsule.

28. The device of claim 27, wherein an inner diameter of the channel is substantially equal to an inner diameter of a distal section of the proximal body of the bushing.

29. The device of claim 28, wherein the lateral extent of the first lateral projection is selected to be substantially equal to the inner diameters of the channel and the distal section of the proximal body of the bushing and wherein a proximal portion of the distal coupling has an inner diameter slightly smaller than that of the channel and the distal section of the proximal body of the bushing so that the first lateral projection becomes lodged against the distal coupling within the bushing.

30. The device of claim 29, wherein the core member includes a second failure point proximal of the first failure point, the core member defining the distal portion distal of the first failure point, a medial portion between the first and second failure points and the proximal section proximal of the second failure point.

31. A method for clipping tissue, comprising:
inserting into a living body to a location adjacent to target tissue to be clipped a distal portion of a flexible elongate member while maintaining a proximal end of the flexible elongate member outside the living body, wherein a control member extends through an applicator and a bushing coupled to a distal end of the elongate flexible member, includes a proximal body coupled to a distal end of the flexible elongate member and a distal coupling movably attached to the proximal body;
positioning a clip coupled to the bushing adjacent to the target tissue, the clip including a capsule releasably coupled to the distal coupling of the bushing via a first coupling member of the capsule hooked over a corresponding first hook of the distal coupling of the bushing, the capsule including a channel extending therethrough, and a pair of clip arms, proximal ends of which are slidably received within the channel;
moving the control member distally relative to the elongate flexible member to move the clip arms to an open configuration, in which distal ends of the clip arms are separated from one another to receive tissue therebetween;
drawing the control member proximally relative to the elongate flexible member to move the clip arms to a closed configuration, in which the distal ends of the clip arms are drawn toward one another so that the target tissue is gripped by the clip arms; and
drawing the control member further proximally to increase a tension applied to a core member coupled to a distal end of the control member and received between and connected to the proximal ends of the clip arms until the tension reaches a predetermined level at which a distal portion of the core member is separated from a proximal portion thereof, wherein a lateral extent of a first lateral projection of the proximal portion of the core member being selected so that, when the distal portion of the core member is separated from the proximal portion of the core member, the first lateral projection is drawn proximally moving the distal coupling into the proximal body of the bushing driving the first coupling member radially outward beyond an outer end of the first hook to free the capsule from the bushing.

32. The method of claim 31, wherein the distal coupling is slidably coupled to the proximal body of the bushing for movement proximally and distally into and out of a distal end of the proximal body.

33. The method of claim 32, wherein an inner diameter of the channel is substantially equal to an inner diameter of a distal section of the proximal body of the bushing.

34. The method of claim 33, wherein the lateral extent of the first lateral projection is selected to be substantially equal to the inner diameters of the channel and the distal section of the proximal body of the bushing and wherein a proximal portion of the distal coupling has an inner diameter slightly smaller than that of the channel and the distal section of the proximal body of the bushing so that the first lateral projection becomes lodged against the distal coupling within the bushing.

35. The method of claim 34, wherein the core member includes a second failure point proximal of a first failure point, the core member defining the distal portion distal of the first failure point, a medial portion between the first and second failure points and a proximal section proximal of the second failure point.
